(19) United States
(12) Patent Application Publication
(10) Pub. No.: US 2021/0220069 A1
(43) Pub. Date: Jul. 22, 2021

(54) SURGICAL DRAPE WITH ACCOMODATIONS FOR IMAGING

(71) Applicant: Nue Medical Solutions, LLC, Aurora, IL (US)

(72) Inventors: Aris John Balderama Arenas, Aurora, IL (US); Chris Chung-Ming Huang, Hinsdale, IL (US)

(21) Appl. No.: 17/147,436

(22) Filed: Jan. 12, 2021

Related U.S. Application Data
(60) Provisional application No. 62/964,595, filed on Jan. 22, 2020.

(51) Int. Cl. A61B 46/10

(52) U.S. Cl. CPC A61B 46/10; A61B 2505/05

(57) ABSTRACT

A surgical drape is provided for use in combination with imaging equipment to allow use of the imaging equipment while keeping a surgical site sterile. The surgical drape includes a main sheet having an incise area and an opening below the incise area. An expandable pocket includes an interior that is in communication with the opening. The expandable pocket includes substantially trapezoid-shaped top and bottom panels with the longer ends of the trapezoids positioned opposite the opening to provide additional pocket material for accommodating the imaging equipment while keeping the opening to a reasonable size.

### CROSS-REFERENCE TO RELATED APPLICATION

This application claims the benefit of and priority to U.S. Provisional Patent Application No. 62/964,595, filed Jan. 22, 2020, the entire contents of which are hereby incorporated by reference.

### BACKGROUND

The present disclosure relates a sterile surgical drape including an expandable region that permits the use of imaging equipment mid-surgery without compromising the sterility of the operating site.

Surgical drapes are known for defining sterile zones in the surgical environment. Surgical drapes may be hung from scaffold-like structure, secured by adhesive, secured by clamps, or secured with tape in a variety of configurations to isolation the patient and the surgical site from the surrounding environment. Some surgeries involve the use of large auxiliary equipment, such as X-ray machines, that present challenges in the surgical environment. These large pieces of equipment must be maneuvered around the patient during a surgery, for example to take X-rays of the surgical site at various times during a procedure, without compromising the sterility of the operating site.

One specific example of such equipment is a C-arm X-ray machine. In a C-arm X-ray machine a large arm in the shape of a C pivots around an axis that is generally aligned with the patient. One end of the X-arm includes the X-ray generator and the other end includes an image intensifier or flat-panel detector. The C arm can be moved around the patient to position the generator and the detector in a desired position relative to the surgical site to confirm proper positioning and placement of the patient's anatomy and/or surgically implanted devices.

Using traditional surgical drapes, it can be extremely difficult to maneuver the C-arm X-ray machine or similarly sized equipment around the surgical site during the surgery without compromising the sterility of the site.

### SUMMARY

In some aspects, a surgical drape is provided for use in combination with imaging equipment. The surgical drape may include a main sheet including a top edge, a bottom edge, and side edges extending between the top edge and the bottom edge. The surgical drape may also include an incise area defined in a central area of the main sheet, an opening below the incise area, and an expandable pocket having an interior in communication with the opening.

The opening may define a width, and the expandable pocket may include a top panel that joins the main sheet along a top pocket edge, and a bottom panel that joins the top panel along an apex edge. The apex edge may be longer than the width. The expandable pocket may include first attachment members positioned along the apex edge, and second attachment members positioned above the top edge. The first attachment members may be securable to the second attachment members to secure the expandable pocket in a collapsed position substantially adjacent the main sheet.

The expandable pocket may include a substantially trapezoidal top panel, a substantially trapezoidal bottom panel joined to the top panel along an apex edge, and a pair of opposed substantially triangular side panels. The top panel may join the main sheet along a top pocket edge, the bottom panel may join the main sheet along a bottom pocket edge, and each side panel may join the main sheet along a respective side pocket edge. The top pocket edge, bottom pocket edge, and side pocket edges may substantially coincide with a perimeter of the opening. The opening may define a width, and the top panel may join the bottom panel along an apex edge. The apex edge may be longer than the width.

The surgical drape may also include weights positioned along the bottom pocket edge. The surgical drape may also include an elastic band extending from one end of the top pocket edge to the apex edge, along the apex edge, and back to an opposite side of the top pocket edge. The expandable pocket may be movable between an expanded position and a collapsed position. The surgical drape may also include pockets secured to the main sheet adjacent the incise area.

### BRIEF DESCRIPTION OF THE DRAWINGS

FIG. 1 is perspective view of an exemplary surgical drape.

FIG. 2 is a top view of the surgical drape of FIG. 1.

FIG. 3 is a physician-side view of the surgical drape of FIG. 1.

FIG. 4 is a patient-side view of the surgical drape of FIG. 1.

FIG. 5 is an end view of the surgical drape of FIG. 1.

FIG. 6 is an end view of the surgical drape of FIG. 1 positioned in a surgical environment and showing movement of the surgical drape when a C-arm X-ray device is extended through the drape.

Before any embodiments of the invention are explained in detail, it is to be understood that the invention is not limited in its application to the details of construction and the arrangement of components set forth in the following description or illustrated in the accompanying drawings. The invention is capable of other embodiments and of being practiced or of being carried out in various ways. Also, it is to be understood that the phraseology and terminology used herein is for the purpose of description and should not be regarded as limiting.

### DETAILED DESCRIPTION

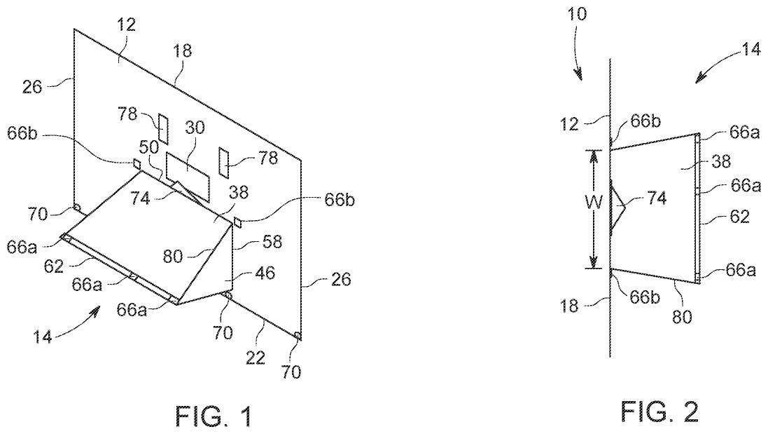

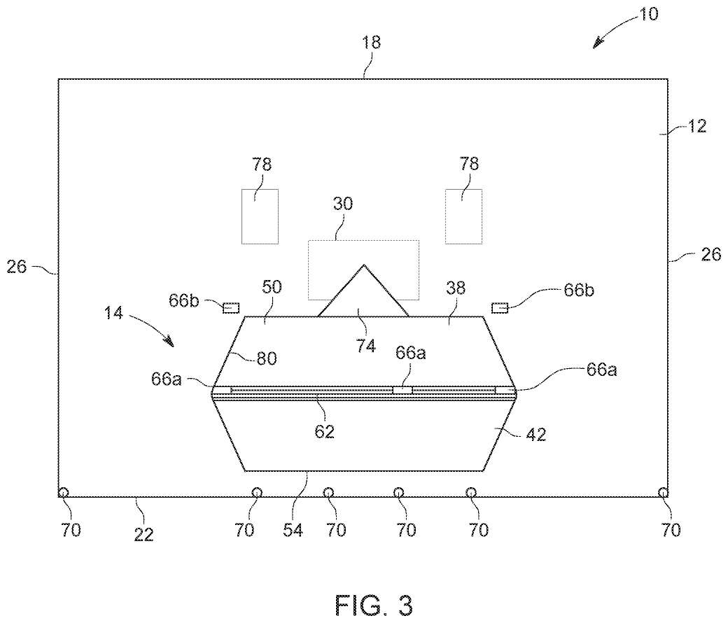

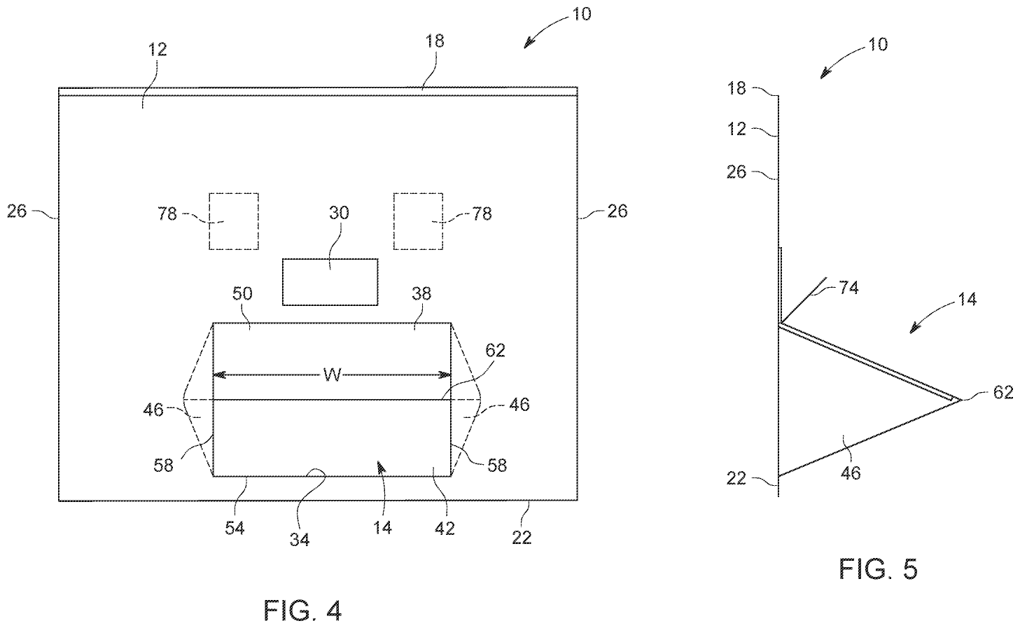

FIGS. 1-6 illustrate a surgical drape 10 defining a main sheet 12 and including an expandable pocket 14 configured to accommodate surgical equipment, such as one end of a C-arm X-ray device while maintaining the sterility of the surgical site. The drape 10 may be formed of a flexible sterile micro-organism proof non-woven plastic material and may include a top edge 18, a bottom edge 22, and side edges 26 extending between the top edge 18 and the bottom edge 22. An incise area 30 may be defined in a central area of the main sheet 10 and may be configured for securement (e.g., via adhesive) to the area around the surgical site, with a portion of the incise area 30 being removable during the surgery to provide access to the site. Below the incise area, the drape 10 defines a substantially rectangular opening 34 (FIG. 4) that communicates with an interior of the expandable pocket 14.

The expandable pocket 14 is illustrated in an expanded position and includes a substantially trapezoid-shaped top panel 38, a substantially trapezoid-shaped bottom panel 42, and a pair of opposed substantially triangular side panels 46. The top panel 38 joins the main sheet 12 along a top pocket edge 50. The bottom panel 42 joins the main sheet along a bottom pocket edge 54, and the side panels 46 join the main sheet along a pair of side pocket edges 58. The top pocket edge 50, bottom pocket edge 54, and side pocket edges 58 substantially coincide with the perimeter of the opening 34 that communicates with the interior of the pocket 14.

The top panel 38 and the bottom panel 42 join one another along an apex edge 62. As shown in FIG. 2, the apex edge 62 is longer than a width W of the opening 34, which increases the amount of material present in the top panel 38 and the bottom panel 42 to more readily accommodate equipment, such as the X-ray arm, during a surgical procedure, as discussed further below. The apex edge 62 is also provided with attachment members 66a near its ends and along its length. The attachment members 66a are configured for attachment to corresponding members 66b provided on the main sheet 12 just above and outboard of the top pocket edge 50. When the attachment members 66a are secured to the attachment members 66b, the pocket 14 transitions to a collapsed position in which the top panel 38 is folded inwardly on itself and covered by the bottom panel 42. The attachment members 66a, 66b may be or include hook and loop closures, magnets, adhesives, and substantial equivalents thereof, suitable to permit repeated movement of the pocket 14 between the expanded position and the collapsed position.

In some embodiments, the drape 10 may include weights 70 along the bottom edge 22 to reduce unwanted movement of the drape during a surgical procedure. The drape 10 may also be provided with a fluid pocket 74 positioned below the incise area 30 to catch fluid and/or discarded instruments or equipment from the procedure. The drape 10 may also include permanent or removeable pockets 78 adjacent the incise area 30 for holding surgical tools or equipment. The drape 10 may also include an elastic member, such as an elastic band 80, extending from one end of the top pocket edge 50, to the apex edge 62, along the apex edge, and back to the opposite end of the top pocket edge 50, to help retain the pocket 14 in the collapsed position and simplify manipulation and movement of the pocket 14.

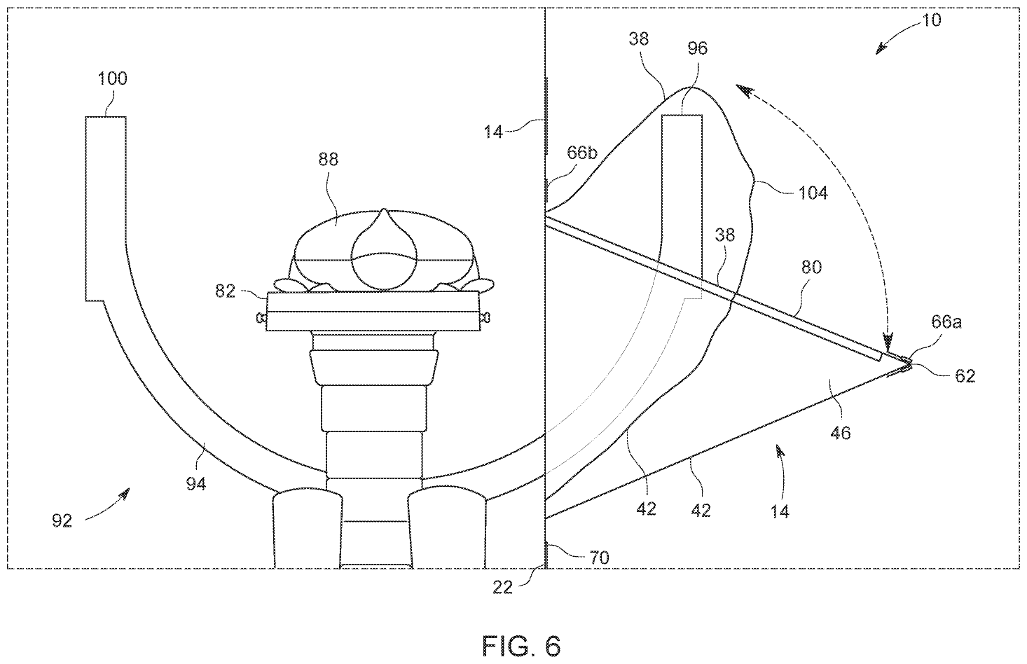

Referring now to FIG. 6, the drape 10 is shown suspended adjacent a surgical table 82 with a patient 88 supported thereon. An X-ray machine 92 including a C-arm assembly 94 is shown extending below the surgical table 82 with a generator end 96 of the machine 92 positioned on one side of the patient 88 and a detector end 100 of the machine 92 positioned on the opposite side of the patient 88. The generator end 96 extends through the opening 34 and into the pocket 14. As shown by the broken line arrow, when the generator end 96 extends into the pocket 14 the pocket 14 moves from the extended position to an accommodating position (indicated by the outline 104) in which the top panel 38 covers the generator end 96 and allows the generator end 96 to be moved to the illustrated position substantially adjacent to the patient 88, for example to take an image of the surgical site. When imaging is complete, the C-arm assembly 94 can be rotated in a clockwise direction (with respect to FIG. 6) to move the generator end 96 to a position below the surgical table 82. When this occurs, the pocket 14 can be returned to the extended position by the surgeon, and subsequently returned to the collapsed position by securing the attachment members 66a to the attachment members 66b. Returning the pocket to the collapsed position folds the top panel 38 inwardly on itself to prevent contamination during further surgical procedures. If additional images are required the surgeon moves the pocket 14 to the expanded position and the C-arm assembly 94 can again be extended through the opening 34 and into the pocket 14.

What is claimed is:

1. A surgical drape for use in combination with imaging equipment, the surgical drape comprising:
a main sheet including a top edge, a bottom edge, and side edges extending between the top edge and the bottom edge;
an incise area defined in a central area of the main sheet;
an opening below the incise area; and
an expandable pocket having an interior in communication with the opening.

2. The surgical drape of claim 1, wherein the opening defines a width, and wherein the expandable pocket includes a top panel that joins the main sheet along a top pocket edge and a bottom panel that joins the top panel along an apex edge, and wherein the apex edge is longer than the width.

3. The surgical drape of claim 1, wherein the expandable pocket includes first attachment members positioned along the apex edge, and the main sheet includes second attachment members positioned above the top edge, and wherein the first attachment members are securable to the second attachment members to secure the expandable pocket in a collapsed position substantially adjacent the main sheet.

4. The surgical drape of claim 1, wherein the expandable pocket includes a substantially trapezoidal top panel, a substantially trapezoidal bottom panel joined to the top panel along an apex edge, and a pair of opposed substantially triangular side panels.

5. The surgical drape of claim 4, wherein the top panel joins the main sheet along a top pocket edge, the bottom panel joins the main sheet along a bottom pocket edge, and each side panel joins the main sheet along a respective side pocket edge, and wherein the top pocket edge, bottom pocket edge, and side pocket edges substantially coincide with a perimeter of the opening.

6. The surgical drape of claim 5, wherein the opening defines a width and the top panel joins the bottom panel along an apex edge, and wherein the apex edge is longer than the width.

7. The surgical drape of claim 5, further comprising weights positioned along the bottom pocket edge.

8. The surgical drape of claim 5, further comprising an elastic band extending from one end of the top pocket edge to the apex edge, along the apex edge, and back to an opposite side of the top pocket edge.

9. The surgical drape of claim 1, wherein the expandable pocket is movable between an expanded position and a collapsed position.

10. The surgical drape of claim 1, further comprising pockets secured to the main sheet adjacent the incise area.
